(19) United States
(12) Patent Application Publication
(10) Pub. No.: US 2023/0201391 A1
(43) Pub. Date: Jun. 29, 2023

(54) STERILISATION APPARATUS FOR PRODUCING PLASMA AND HYDROXYL RADICALS

(71) Applicant: Creo Medical Limited, Chepstow, South Wales (GB)

(72) Inventors: Christopher Paul HANCOCK, Chepstow (GB); George Christian ULLRICH, Bethesda (GB); David Edward WEBB, Bethesda (GB)

(21) Appl. No.: 17/927,268

(22) PCT Filed: May 28, 2021
(86) PCT No.: PCT/EP2021/064411

§ 371 Date: Nov. 22, 2022

(30) Foreign Application Priority Data
Jun. 1, 2020 (GB) 2008156.8

(51) Int. Cl. A61L 2/14; A61L 2/26; H05H 1/46

(52) U.S. Cl. CPC A61L 2/14; A61L 2/26; H05H 1/4637; A61L 2202/11; A61L 2202/122; A61L 2202/123; H05H 2245/20; H05H 2245/36

(57) ABSTRACT

Sterilisation systems suitable for clinical use, e.g. on the human body, medical apparatuses, or hospital bed spaces. The sterilisation apparatus comprises: a microwave source arranged to generate microwave energy; a mist generator arranged to generate a flow of water mist; a gas supply; a manifold connected to receive the flow of water mist from the mist generator; and a plurality of plasma applicators connected to the manifold, wherein each plasma applicator is connected to receive microwave energy from the microwave source and a flow of gas from the gas supply, wherein each plasma applicator is configured to strike a plasma at a distal end thereof, wherein the distal ends of the plurality of plasma applicators are disposed in a plasma generating region defined by the manifold, and wherein the manifold is configured to direct the flow of water mist through the plasma generating region to an outlet thereof.

### FIELD OF THE INVENTION

The invention relates to sterilisation systems suitable for clinical use, e.g. on the human body, medical apparatuses, or hospital bed spaces. For example, the invention may provide a system that can be used to destroy or treat certain bacteria and/or viruses associated with the human or animal biological system and/or the surrounding environment. This invention is particularly useful for sterilising or decontaminating enclosed or partially enclosed spaces.

### BACKGROUND TO THE INVENTION

Bacteria are single-celled organisms that are found almost everywhere, exist in large numbers and are capable of dividing and multiplying rapidly. Most bacteria are harmless, but there are three harmful groups; namely: cocci, spirilla, and bacilla. The cocci bacteria are round cells, the spirilla bacteria are coil-shaped cells, and the bacilli bacteria are rod-shaped. The harmful bacteria cause diseases such as tetanus and typhoid.

Viruses can only live and multiply by taking over other cells, i.e. they cannot survive on their own. Viruses cause diseases such as colds, flu, mumps and AIDS. Viruses may be transferred through person-to-person contact, or through contact with region that is contaminated with respiratory droplets or other virus-carrying bodily fluids from an infected person.

Fungal spores and tiny organisms called protozoa can cause illness.

Sterilisation is an act or process that destroys or eliminates all form of life, especially micro-organisms. During the process of plasma sterilisation, active agents are produced. These active agents are high intensity ultraviolet photons and free radicals, which are atoms or assemblies of atoms with chemically unpaired electrons. An attractive feature of plasma sterilisation is that it is possible to achieve sterilisation at relatively low temperatures, such as body temperature. Plasma sterilisation also has the benefit that it is safe to the operator and the patient.

Plasma typically contains charged electrons and ions as well as chemically active species, such as ozone, nitrous oxides, and hydroxyl radicals. Hydroxyl radicals are far more effective at oxidizing pollutants in the air than ozone and are several times more germicidal and fungicidal than chlorine, which makes them a very interesting candidate for destroying bacteria or viruses and for performing effective decontamination of objects contained within enclosed spaces, e.g. objects or items associated with a hospital environment.

OH radicals held within a “macromolecule” of water (e.g. a droplet within a mist or fog) are stable for several seconds and they are 1000 times more effective than conventional disinfectants at comparable concentrations.

An article by Bai et al titled “Experimental studies on elimination of microbial contamination by hydroxyl radicals produced by strong ionisation discharge” (Plasma Science and Technology, vol. 10, no. 4, August 2008) considers the use of OH radicals produced by strong ionisation discharges to eliminate microbial contamination. In this study, the sterilisation effect on E. coli and B. subtilis is considered. The bacteria suspension with a concentration of 10⁷ cfu/ml (cfu=colony forming unit) was prepared and a micropipette was used to transfer 10 μl of the bacteria in fluid form onto 12 mm×12 mm sterile stainless steel plates. The bacteria fluid was spread evenly on the plates and allowed to dry for 90 minutes. The plates were then put into a sterile glass dish and OH radicals with a constant concentration were sprayed onto the plates. The outcomes from this experimental study were:

1. OH radicals can be used to cause irreversible damage to cells and ultimately kill them;

2. The threshold potential for eliminating micro-organisms is ten thousandths of the disinfectants used at home or abroad;

3. The biochemical reaction with OH is a free radical reaction and the biochemical reaction time for eliminating micro-organisms is about 1 second, which meets the need for rapid elimination of microbial contamination, and the lethal time is about one thousandth of that for current domestic and international disinfectants;

4. The lethal density of OH is about one thousandths of the spray density for other disinfectants—this will be helpful for eliminating microbial contamination efficiently and rapidly in large spaces, e.g. bed-space areas; and

5. The OH mist or fog drops oxidize the bacteria into CO₂, H₂O and micro-inorganic salts. The remaining OH will also decompose into H₂O and O₂, thus this method will eliminate microbial contamination without pollution.

WO 2009/060214 discloses sterilisation apparatus arranged controllably to generate and emit hydroxyl radicals. The apparatus includes an applicator which receives RF or microwave energy, gas and water mist in a hydroxyl radical generating region. The impedance at the hydroxyl radical generating region is controlled to be high to promote creation of an ionisation discharge which in turn generates hydroxyl radicals when water mist is present. The applicator may be a coaxial assembly or waveguide. A dynamic tuning mechanism e.g. integrated in the applicator may control the impedance at the hydroxyl radical generating region. The delivery means for the mist, gas and/or energy can be integrated with each other.

WO 2019/175063 discloses a sterilisation apparatus that uses thermal or non-thermal plasma to sterilise or disinfect surgical scoping devices. In one example, a plasma generating region is formed at a distal end of a coaxial transmission line, which convey RF or microwave energy to strike and sustain the plasma. A gas passageway is formed around an outer surface of the coaxial transmission line. The gas passageway is in fluid communication with the plasma generating region through notches in a cylindrical electrode mounted on a distal end of the coaxial transmission line. In some examples, water through a passageway formed within the inner conductor of the coaxial transmission line, from where it is sprayed on to the surface of an object before the plasma passes over it.

### SUMMARY OF THE INVENTION

At its most general, the invention provides a sterilisation apparatus suitable for generating hydroxyl radicals for sterilising an enclosed space, in which energy, gas and water mist feeds are combined in a manner that permit the apparatus to be readily scaled to the size of enclosure. In particular, the sterilisation apparatus provides a manifold in which a plurality of plasma applicators may be mounted around a plasma generating region to form a ring-shaped plasma arc through which a flow of water mist is directed to form the hydroxyl radicals.

According to one aspect, the invention provides sterilisation apparatus comprising: a microwave source arranged to generate microwave energy; a mist generator arranged to generate a flow of water mist; a gas supply; a manifold connected to receive the flow of water mist from the mist generator; and a plurality of plasma applicators connected to the manifold, wherein each plasma applicator is connected to receive microwave energy from the microwave source and a flow of gas from the gas supply, wherein each plasma applicator is configured to strike a plasma at a distal end thereof, wherein the distal ends of the plurality of plasma applicators are disposed in a plasma generating region defined by the manifold, and wherein the manifold is configured to direct the flow of water mist through the plasma generating region to an outlet thereof. In use, the manifold receives a flow of water mist that is directed through a plasma generating region in which plasma created using a plurality of plasma applicators is present. The mechanism for plasma generation is independent of the water mist delivery. This means that the plasma applicators do not need to be adapted to accommodate a flow of mist. Moreover, it permits the apparatus to be scalable both in terms of the size of the plasma generating region (controlled by the number of plasma applicators) and in terms of the flow rate (volume per second) of water mist. The manifold may be adapted to combine together water mist inputs from multiple mist generators as well as receiving a plurality of plasma applicators.

The manifold may comprise a hollow body that acts as a fluid flow conduit from one or more inlets to the outlet. For example, the manifold may define a flow direction of the water mist from an inlet thereof to the outlet. The flow direction may be aligned with the direction of the flow of water mist that is received into the manifold. That is, the water mist is substantially undeflected as it travels through the manifold. This may be advantageous in obtaining a large sterilisation range for a given water mist flow rate.

The manifold may be made (e.g. moulded) from an electrically insulating material so that it does not interfere with the delivery of the microwave energy.

Each plasma applicator may extend transversely to the flow of water mist through the plasma generating region. For example, the manifold may comprise a plurality of lateral ports (i.e. ports in a side surface thereof) to receive the plasma applicators. With this arrangement, the direction in which energy is injected into the plasma generating region may thus be orthogonal to the flow of water mist.

The plurality of plasma applicators may comprise one or more pairs of plasma applicators that face one another on opposing sides of the plasma generating region. The plasma generating region may comprise or consist of a space between the one or more pairs of plasma applicators. The plurality of plasma applicators may be arranged around the plasma generating region in a manner that causes their respective plasma arcs to combine to form a ring.

Each plasma applicator may be configured to strike a plasma using the microwave energy only. However, in other embodiments the apparatus may include an RF source arranged to supply a pulse of RF energy to strike the plasma, with the microwave energy used to sustain it. An example of an RF strike and microwave sustain set up is given in WO 2019/175063.

In an arrangement capable of striking the plasma using microwave energy only, each plasma applicator may comprise: a conductive tube; and an elongate conductive member extending along a longitudinal axis of the conductive tube. The conductive tube and elongate conductive member may provide a first coaxial transmission line at a proximal end of the plasma applicator, and a second coaxial transmission line at a distal end of the plasma applicator. The first coaxial transmission line may be configured as a quarter wavelength impedance transformer. The quarter wavelength impedance transform may operate to transform a first impedance (e.g. of a coaxial cable that feeds the plasma applicator) to a second impedance (e.g. the impedance of the second coaxial transmission line). The second coaxial transmission line may be configured with a higher impedance than the first coaxial transmission line. An impedance of the first and second coaxial transmission lines may be determined by the geometry of the structure, e.g. the relative size of the diameter of the elongate conductive member and the inner diameter of the conductive tube. The second coaxial transmission line may have an impedance selected to establish an electric field at its distal end that is suitable to strike a plasma in the gas that flows through the plasma applicator. The flow of gas received by each plasma applicator may pass between the conductive tube and elongate conductive member, where it also acts as a dielectric (insulating) material of the first and second coaxial transmission lines.

A sleeve of insulating material, e.g. quartz or the like, may be mounted in a distal end of the conductive tube. The sleeve may assist in focusing the electric field at the distal end of the second coaxial transmission line, thereby facilitating the plasma strike at a desired location.

Each plasma applicator may comprise a gas inlet tube configured to deliver the flow of gas to a space between the conductive tube and the elongate conductive member. The gas inlet tube may extend transversely to the longitudinal axis of the conductive tube.

Each plasma applicator may comprise a proximal connector configured to connect to a coaxial cable conveying the microwave energy from the microwave source. The proximal connector may be configured to electrically connect an inner conductor of the coaxial cable to the elongate conductive member, and to electrically connect an outer conductor of the coaxial cable to the conductive tube. The microwave energy may thus be delivered in line with the longitudinal axis of the conductive tube, which may assist in efficient coupling. Meanwhile, the gas inlet tube may be arranged transversely to the longitudinal axis, which may be advantageous because it does not interfere with the delivery of the microwave energy.

The microwave source may be generator capable of producing microwave energy having a power suitable for striking a plasma. In one example, the microwave source comprises a magnetron. The microwave source may further comprise a waveguide to coaxial adaptor to couple energy from the magnetron into one or more coaxial cables which connect to the plurality of plasma applicators. In other examples, the microwave source may comprise an oscillator and a power amplifier.

The mist generator may comprise any suitable means for generating a mist of water droplets or water vapour. For example, the mist generator may be an ultrasonic fogging device in which ultrasonic vibrations are applied to a water source to generate fine water droplets. In another example, the mist generator may operate to heat water to produce water vapour.

The apparatus may comprise a plurality of mist generators, wherein the manifold comprises a plurality of inlet ports, each inlet port being connectable to a respective mist generator. The apparatus may thus be scalable by adapting the manifold to receive a desired number of mist generator inputs.

The gas supply may be connected to deliver a gas flow to the or each mist generator. The gas flow may entrain water mist formed by the mist generator to create the flow of water mist. In this way, the flow rate of the mist may be controllable. This may be particularly desirable if there are a plurality of mist generators, where it may be useful to be able to independently control the gas flow rate for each mist generator, e.g. in order to ensure that a uniform flow is received within the manifold.

Preferably the gas supply is a supply of argon gas. However, any other suitable gas may be chosen, e.g. carbon dioxide, helium, nitrogen, a mixture of air and any one of these gases, for example 10% air/90% helium.

The sterilisation apparatus may be configured for use with an enclosure. For example, the outlet of the manifold may be couplable to an enclosure, such as a box, room, vehicle or the like. The enclosure may define a space to be sterilised. The apparatus may be scaled to the size of the enclosure. For example, the number of mist generators, the flow rate of gas, and the number of plasma applicators and all factors that can be adapted depending on the enclosure. By providing a manifold capable of combining inputs from multiple individual components, the apparatus of the invention facilitates the ability to adapt to different environments.

Herein, the term “inner” means radially closer to the centre (e.g. axis) of the coaxial cable, probe tip, and/or applicator. The term “outer” means radially further from the centre (axis) of the coaxial cable, probe tip, and/or applicator.

The term “conductive” is used here to mean electrically conductive, unless the context dictates otherwise.

Herein, the terms “proximal” and “distal” refers to the ends of the applicator. In use, the proximal end is closer to a generator for providing the RF and/or microwave energy, whereas the distal end is further from the generator.

In this specification “microwave” may be used broadly to indicated a frequency range of 400 MHz to 100 GHz, but preferably in the range 1 GHz to 60 GHz. Specific frequencies that have been considered are: 915 MHz, 2.45 GHz, 3.3 GHz, 5.8 GHz, 10 GHz, 14.5 GHz, and 25 GHz. In contrast, this specification uses “radiofrequency” or “RF” to indicate a frequency range that is at least three orders of magnitude lower, e.g. up to 300 MHz, preferably 10 kHz to 1 MHz, and most preferably 400 kHz. The microwave frequency may be adjusted to enable the microwave energy delivered to be optimised. For example, a probe tip may be designed to operate at a certain frequency (e.g. 900 MHz), but in use the most efficient frequency may be different (e.g. 866 MHz).

### BRIEF DESCRIPTION OF THE DRAWINGS

Features of the invention are now explained in the detailed description of examples of the invention given below with reference to the accompanying drawings, in which:

FIG. 1 is a schematic diagram of a sterilisation apparatus according to an embodiment of the present invention;

FIG. 2 is a schematic top view of a feed manifold suitable for use with the sterilisation apparatus of FIG. 1;

FIG. 3 is a schematic front view of the feed manifold of FIG. 2;

FIG. 4 is a schematic side view of a plasma applicator that is suitable for use with the sterilisation apparatus of FIG. 1; and

FIG. 5 is a schematic cross-sectional view of the plasma applicator of FIG. 4.

### DETAILED DESCRIPTION; FURTHER OPTIONS AND PREFERENCES

This invention relates to a device for performing sterilisation using hydroxyl radicals that are generated by creating a plasma in the presence of water mist.

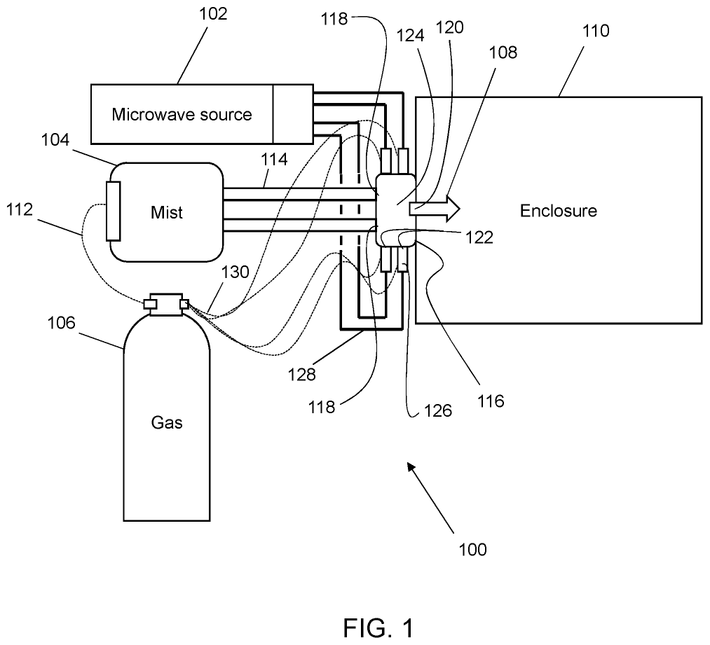

FIG. 1 is a schematic view of a sterilisation apparatus 100 that is an embodiment of the invention. The sterilisation apparatus 100 operates to combine feeds from each of a microwave source 102, a mist generator 104 and a gas supply 106 to generate a flow 108 of hydroxyl radicals into an enclosure 110 to be sterilised.

The microwave source 102 may be any suitable microwave generator for outputting microwave energy, i.e. electromagnetic energy having a frequency in a range of 400 MHz to 100 GHz, preferably in the range 1 GHz to 60 GHz. For example, it may be a magnetron arranged output microwave energy having a frequency of 2.45 GHz. In other embodiments, the microwave source may comprise an oscillator and power amplifier. The microwave source 102 may be configured to output microwave energy with a power equal to or greater than 200 W, preferably 500 W or more, e.g. 800 W or the like.

The mist generator 104 may comprise one or more ultrasonic fogging devices, in which a fine mist of water droplets is obtained by applying ultrasonic energy to a vessel storing liquid water, e.g. distilled water. Alternatively, the mist generator 104 may comprise a device for generating water vapour (steam) by applying heat to stored water.

The gas supply 106 may comprise a canister of pressurised inert gas, such as argon, nitrogen, carbon dioxide or the like. Alternatively, the sterilisation apparatus may operate with air as the gas medium in which a plasma is struck. In this example, the gas supply may comprise a fan or other means for generating a directable gas flow.

In this example, the gas supply 106 has a first connection 112 through which a first gas flow is supplied to the mist generator 104. The first gas flow entrains the mist or water vapour from the mist generator 104 and conveys it through mist conduits 114 towards the enclosure 110. Where there are multiple mist generators 104, the first connection 112 may have multiple branches, and there may be multiple mist conduits 114.

The enclosure 110 may be any space that requires sterilisation. It may be a box or room (e.g. operating theatre or hospital suite) or a vehicle interior (e.g. an ambulance or the like). The flow rate from the apparatus into the enclosure 110 may be adjustable, e.g. to facilitate the spread of hydroxyl radicals within the enclosed volume.

The sterilisation apparatus 100 further comprises a manifold 116 that is configured to combine the microwave energy, mist and gas to generate the flow 108 of hydroxyl radicals. In this embodiment, the manifold 116 defines an internal volume that operates as a plasma generating region 124 in a manner discussed in more detail below. The manifold 116 comprises a plurality of proximal inlet ports 118 connected to the mist conduits 114 and an outlet port 120 through which the flow 108 of hydroxyl radicals passes into the enclosure 110. The inlet ports 118 feed into the plasma generating region 124. The outlet port 120 is an exit aperture of the plasma generating region 124. The inlet ports 118 may be aligned with the outlet port 120 in the sense that the flow of mist from the mist conduits 114 enters the manifold 116 in a direction that is aligned with, e.g. parallel to, the direction in the which the flow 108 of hydroxyl radicals exits the manifold 116.

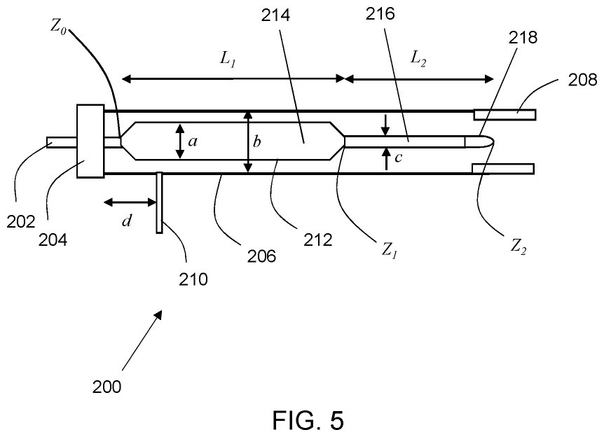

The manifold 116 further comprises a plurality of lateral ports 122 that are disposed on either side of the plasma generating region 124. In this example there are a pair of lateral ports 122 arranged on opposing sides of the manifold 116. Each lateral port 122 is configured to receive a plasma applicator 126. Each plasma applicator 126 is connected to receive microwave energy from the microwave source 102, e.g. via a respective coaxial cable 128 or the like. As discussed below in more detail with reference to FIGS. 4 and 5, each plasma applicator 126 is configured to create an electric field at a distal end thereof that is capable of striking a plasma in the gas that flows through the manifold 116. Each plasma applicator 126 extends through its respective lateral port 122 so that its distal end lies within the plasma generating region 124.

In this example, the gas supply 106 further comprises a second connection 130 that provides a separate gas feed to each of the plasma applicators 126. Where there are a plurality of plasma applicators 126, the second connection 130 may comprise a plurality of branches. With this arrangement, gas enters the plasma generating region 124 from both the mist conduits 114 and from the plasma applicators 126.

In use, gas is supplied through both the first connection 112 and the second connection 130. Mist is created by the mist generator 104 and entrained in the gas from the first connection 112, whereupon it flows though the mist conduits 114 into the manifold 116. Meanwhile gas flows from the second connection 130 through the plasma applicators 126 to enter the plasma generating region 124. Microwave energy supplied from the microwave source 102 creates an electric field within the plasma generating region 124 to strike a plasma in the gas. The plasma applicators 126 may be disposed around the plasma generating region 124 in a manner that ring-like plasma arc is visible in the outlet port 120.

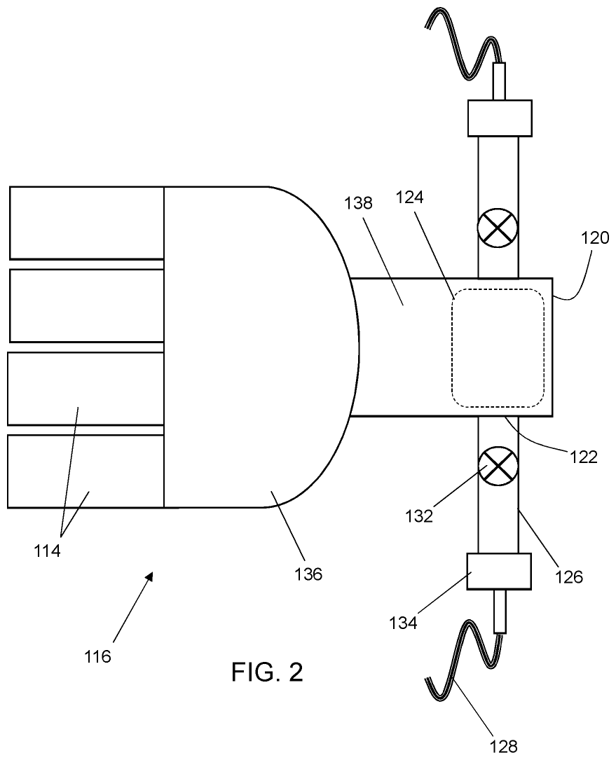

FIG. 2 is a schematic top view of a manifold 116 that can be used an embodiment of the invention. Features already discussed are provided with the same reference numbers, and description thereof is not repeated. In this example, four mist conduits 114 are received at a proximal side of a funnel element 136, which acts to combine the flows from each mist conduit 114 into a single tube 138, which extends from a distal side of the funnel element 136. The plasma generating region 124 is formed within the tube 138. The outlet port 120 that leads to the enclosure (not shown) is at the distal end of the tube 138.

Similarly, the lateral ports 122 through which the plasma applicators 126 extend into the plasma generating region 124 are formed in side surfaces of the tube 138. Each plasma applicator 126 comprises a proximal connector 134 that is connectable to the coaxial cable 128. As discussed above, each plasma applicator 126 has a dedicated gas feed, which enters though an gas inlet tube 132. The gas inlet tube 132 extends into a direction that is transverse to the direction in which the plasma applicator 126 extends into the plasma generating region 124. In FIG. 2 the direction of the gas inlet tube 132 is into the page.

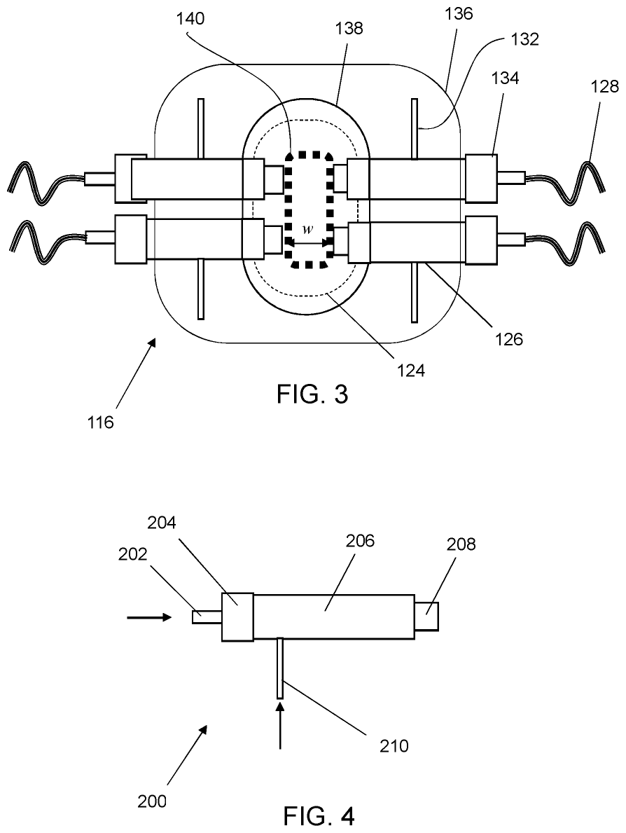

FIG. 3 shows a front view of the manifold 116 shown in FIG. 2. Features already discussed are provided with the same reference numbers, and description thereof is not repeated. In this example, there are two plasma applicators 126 on each side of the plasma generating region 124, disposed one on top of the other. In this view, the portions of the plasma applicators 126 that extends into the tube 138 are visible through the outlet port. The opposing plasma applicators 126 are spaced by a distance w, which in this example is 3 mm, but may be selected at a scale appropriate to the size of plasma arc created by the combination of gas flow rate and level of microwave energy supplied. The plasma ring created in operation is shown schematically by dotted line 140. It may be seen that flow of mist from the mist conduits passes through and around the plasma ring, which thereby causes the formation of hydroxyl radicals in the gas flow to facilitate sterilisation.

FIG. 4 is a side view of a plasma applicator 200 that can be used in the apparatus discussed above. The plasma applicator 200 is a generally elongate cylindrical member, defined by a conductive tube 206, e.g. of copper or the like. A connector 204 is mounted at a proximal end of the conductive tube 206 to receive a coaxial cable 202. Microwave energy conveyed along the coaxial cable 202 can therefore be delivered into the conductive tube 206 in a direction in line with a longitudinal axis of the conductive tube 206. The conductive tube 206 is open at its distal end. A gas feed tube 210 is mounted on a side of the conductive tube 206 towards its proximal end. The gas feed tube 210 defines a flow path that passes into an internal volume of the conductive tube 206. The flow path is angled relative to the axis of the conductive tube 206. In this example, the flow path lies transverse to that axis. Gas delivered through the gas feed tube 210 flows through the conductive tube 206 to exit at its distal end. A quartz tube 208 in mounted coaxially with the conductive tube 206 in the distal end thereof. The quartz tube 208 protrudes beyond the distal end of the conductive tube 108, and overlaps with an inner surface of the conductive tube along a distal length thereof, as shown in FIG. 5.

FIG. 5 is a schematic cross-sectional view through the plasma applicator 200 shown in FIG. 4. The plasma applicator 200 comprises an elongate conductive member 212 extending coaxially with the conductive tube 260 through the internal volume. A proximal end of the elongate conductive member 212 is connected to the inner conductor of the coaxial cable 202. The elongate conductive member 212 has a proximal portion 214 and a distal portion 216 with differing diameters. In this example, the proximal portion 214 has a diameter a that is larger than a diameter c of the distal portion 216. The distal portion 216 terminates at a distal tip 218, which is rounded in this example. In conjunction with the conductive tube 206, the proximal portion 214 and distal portion 216 respectively define a first coaxial transmission line and a second coaxial transmission line.

The plasma applicator 200 includes a quarter wave transformer arranged to increase the impedance at the distal tip thereof to facilitate a plasma strike with delivered microwave energy. The quarter wave transformer may be provided by the first coaxial transmission line defined above, i.e. by the conductive tube 206 and proximal portion 214 of the elongate conductive member 212.

The operation of the quarter wavelength transformer is now explained. The coaxial cable 202 may be of any conventional type, and is indicated in FIG. 5 as having an impedance of Z₀, which may be 50Ω. An outer conductor of the coaxial cable is electrically connected to the conductive tube 206, which has a uniform inner diameter b along its length. An inner conductor of the coaxial cable 202 is electrically connected to the elongate conductive member 212.

An impedance Z_{L}_{₁} of the first coaxial transmission line can be expressed as:

$${Z}_{{L}_{1}} = \frac{138}{\sqrt{{ε}_{r}}} {log}_{10} \frac{b}{a}$$

An impedance Z_{L}_{₂} of the second coaxial transmission line can be expressed as:

$${Z}_{{L}_{2}} = \frac{138}{\sqrt{{ε}_{r}}} {log}_{10} \frac{b}{c}$$

The first coaxial transmission line has a length L₁, and the second coaxial transmission line has a length L₂. Both L₁ and L₂ are arranged to be an odd multiple of a quarter wavelength of the microwave energy conveyed by the coaxial cable 202. For example, where the microwave energy has a frequency of 2.45 GHz, the L₁ and L₂ may be 30.6 mm, so the plasma applicator itself has an overall length of 6-8 cm.

Consequently, an impedance Z₁ of the junction of the first coaxial transmission line and the second coaxial transmission line can be expressed as:

$${Z}_{1} = \frac{{( {Z}_{{L}_{1}} )}^{2}}{{Z}_{0}}$$

And an impedance Z₂ at the distal tip 218 the second coaxial transmission line can be expressed as:

$${Z}_{2} = \frac{{( {Z}_{{L}_{2}} )}^{2}}{{Z}_{1}}$$

Substituting and simplifying the above expressions permits Z₂ to be expressed as:

$$( \frac{{( {log}_{10} b - {log}_{10} c )}^{2}}{{( {log}_{10} b - {log}_{10} a )}^{2}} ) {Z}_{0}$$

For an input power P at the proximal end of the plasma applicator 200, and assuming minimal loss of energy along the first and second coaxial transmission lines, a voltage V at the distal tip may be expressed as:

V=\sqrt{PZ₂}=M\sqrt{PZ₀}

wherein M is a voltage multiplication factor equal to

$$\sqrt{\frac{{( {log}_{10} b - {log}_{10} c )}^{2}}{{( {log}_{10} b - {log}_{10} a )}^{2}}}$$

In one example, the dimensions for the plasma applicator 200 may be as follows: a=6.5 mm, b=12.5 mm, c=1 mm. This yields a voltage multiplication factor equal to 3.862. For Z₀=50Ω and an input power P=250 W, this yields a voltage at the distal tip 218 of 431.8 V. It can therefore be understood that this structure is effective in yielding a voltage that can provide an electric field at the distal end of the applicator that is high enough to cause electrical breakdown of gas conveyed through the conductive tube 206.

In FIG. 5, the gas feed tube 210 is located at a distance d from a proximal end of the conductive tube 206. The distance d may be selected to ensure that the gas feed tube does not affect the transmission of microwave energy by the first coaxial transmission line and the second coaxial transmission line. In one example, the distance d is 15 mm.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A sterilisation apparatus comprising:
a microwave source arranged to generate microwave energy;
a mist generator arranged to generate a flow of water mist;
a gas supply;
a manifold connected to receive the flow of water mist from the mist generator; and
a plurality of plasma applicators connected to the manifold,
wherein each plasma applicator is connected to receive microwave energy from the microwave source and a flow of gas from the gas supply,
wherein each plasma applicator is configured to strike a plasma at a distal end thereof,
wherein the distal ends of the plurality of plasma applicators are disposed in a plasma generating region defined by the manifold, and
wherein the manifold is configured to direct the flow of water mist through the plasma generating region to an outlet thereof.

2. The sterilisation apparatus of claim 1, wherein a flow direction of the water mist from an inlet of the manifold to the outlet thereof is aligned with the direction of the flow of water mist that is received into the manifold.

3. The sterilisation apparatus of claim 1, wherein each plasma applicator extends transversely to the flow of water mist through the plasma generating region.

4. The sterilisation apparatus of claim 1, wherein the plurality of plasma applicators comprise one or more pairs of plasma applicators that face one another on opposing sides of the plasma generating region.

5. The sterilisation apparatus of claim 1, wherein each plasma applicator comprises:
a conductive tube; and
an elongate conductive member extending along a longitudinal axis of the conductive tube,
wherein the conductive tube and elongate conductive member provide a first coaxial transmission line at a proximal end of the plasma applicator, and a second coaxial transmission line at a distal end of the plasma applicator, and
wherein the first coaxial transmission line is configured as a quarter wavelength impedance transformer.

6. The sterilisation apparatus of claim 5, wherein the second coaxial transmission line is configured with a higher impedance than the first coaxial transmission line.

7. The sterilisation apparatus of claim 5, wherein the flow of gas received by each plasma applicator passes between the conductive tube and elongate conductive member.

8. The sterilisation apparatus of claim 7, wherein each plasma applicator comprises a gas inlet tube configured to deliver the flow of gas to a space between the conductive tube and the elongate conductive member, wherein the gas inlet tube extends transversely to the longitudinal axis of the conductive tube.

9. The sterilisation apparatus of claim 5, wherein each plasma applicator comprises a proximal connector configured to connect to a coaxial cable conveying the microwave energy from the microwave source, wherein the proximal connector is configured to electrically connect an inner conductor of the coaxial cable to the elongate conductive member, and to electrically connect an outer conductor of the coaxial cable to the conductive tube.

10. The sterilisation apparatus of claim 1, wherein the microwave source comprises a magnetron.

11. The sterilisation apparatus of claim 1, wherein the mist generator comprises an ultrasonic fogging device.

12. The sterilisation apparatus of claim 1 comprising a plurality of mist generators, wherein the manifold comprises a plurality of inlet ports, each inlet port being connectable to a respective mist generator.

13. The sterilisation apparatus of claim 1, wherein the gas supply is connected to deliver a gas flow to the mist generator, wherein the gas flow entrains water mist formed by the mist generator to create the flow of water mist.

14. The sterilisation apparatus of claim 1, wherein the gas is argon, nitrogen or carbon dioxide.

15. The sterilisation apparatus of claim 1, wherein the outlet of the manifold is couplable to an enclosure that defines a space to be sterilised.
